SEQUENCE LISTING (2 sequences)

SEQ ID NO: 1 - DNA, 1540 bp, Corynebacterium glutamicum
  CDS (230)..(1294)
tgaccagctcgaggctctcggctacatcccagttaccggcgagcactacgatcgcctcgttgcagcagttgaagcaattcagtaataaaccgctgccgtagcttcacgaaaccagtgtgaagttgcactttaagtgaatcgggcgccctctcccagcaattgggggtagggcgcccgattttcctaacaagctttttatcaatacgccagttaaggaaataaaccaccaatggccactaatgagtcagtctcggagaagcaacgcctggatgcaaccagggtgcaggcacatcctgtagcagttaatgcgaactcctctcagaccaagccttcaaagaagattgtcgccgaaggtggcggaagcgttaagcgtcccggcgatcgcatcttcgaagtcctatccaccgcttctgcagcaatcattactgcgataatcattgccattgcggcgttccttatctggcgtgctgttcccgccttgatgcgaaatgctgaaggtattggcggattcttcacttattcaggcgcttggaacaccaccgacattgatgcaatgtacttcggtattccaaacctgctagctgcaacacttctcatctctgtcatcgcactgatcatcgccatgccgattgctcttggtattgcgatcttcttgtccaactactcaccaaagcgcctggttaagccacttggctacatggtggacatgctggctgctgtgccttccatcgtctacggcctttggggctggcaggtgctcggaccagctctgtccggtttctacacctggattgaaagctggggcggaagcttcttcctcttcgctacttaccaaaactcaccttcttttgctaccggccgtaacatgctcaccggtggcatcgtgctcgcagtgatgatccttcctgttatcgcagcaaccgcacgtgaagttttcatacagactccaaagggccacattgaatctgctcttgcacttggcgcaacccgctgggaagtcgttcgtttgacggttctcccattcggaatgtccggctacgtttccggcgcgatgctcggcctcggccgcgcactgggtgagaccatggcgctatacatggttgtttctccatcctcggcgttccgcttctcgcttttcgatggcggtaccaccttcgcaacggccatcgccaatgccgctccagaattcaacgacaacacccgcgcaggcgcgtacatctccgccggcctcgtgctgttcgcccttaccttcatcgtcaacgctggcgctcgcgccatggttaaccgcggaaagtagaaggggacaaaatcatgactaacaatgttgttactccgcgcatggatgagcctttaaagaagagctcagccttcacgacatctcctccagccgtaagaccaccaacaccgcagcaaccgtcatcatttatggtgcgatgctcatcgcagctgtgccactggtttgggtgctgtggaccgtgatctctcgaggcatcgctccgatcctcactgctgattggtggtccacctcccaggctggcgt

SEQ ID NO: 2 - protein, 355 aa, Corynebacterium glutamicum
MATNESVSEKQRLDATRVQAHPVAVNANSSQTKPSKKIVAEGGGSVKRPGDRIFEVLSTASAAIITAIIIAIAAFLIWRAVPALMRNAEGIGGFFTYSGAWNTTDIDAMYFGIPNLLAATLLISVIALIIAMPIALGIAIFLSNYSPKRLVKPLGYMVDMLAAVPSIVYGLWGWQVLGPALSGFYTWIESWGGSFFLFATYQNSPSFATGRNMLTGGIVLAVMILPVIAATAREVFIQTPKGHIESALALGATRWEVVRLTVLPFGMSGYVSGAMLGLGRALGETMALYMVVSPSSAFRFSLFDGGTTFATAIANAAPEFNDNTRAGAYISAGLVLFALTFIVNAGARAMVNRGK

(19) United States
(12) Patent Application Publication
(10) Pub. No.: US 2002/0106751 A1
(43) Pub. Date: Aug. 8, 2002

(54) Nucleotide sequences coding for the pstC2 gene

(76) Inventors: Mike Farwick, Bielefeld (DE); Klaus Huthmacher, Gelnhausen (DE); Walter Pfefferle, Halle (DE); Jennifer Brehme, Bielefeld (DE)

Correspondence Address: SMITH, GAMBRELL & RUSSEL,LLP, Suite 800, 1850 M Street. N.W., Washington, DC, 20036, US

(21) Appl. No.: 09/951,769

(22) Filed: Sep. 14, 2001

(30) Foreign Application Priority Data
Sep. 14, 2000 (DE) 100 45 486.0

(51) Int. Cl. C12P 13/04; C07H 21/04; C12N 9/10; C12N 1/21; C12P 21/02; C12N 15/74

(52) U.S. Cl. 435/106; 435/252.3; 435/193; 435/69.1; 435/320.1; 536/23.2

(57) ABSTRACT

The invention relates to an isolated polynucleotide having a polynucleotide sequence which codes for the pstC2 gene, and a host-vector system having a coryneform host bacterium in which the pstC2 gene is present in attenuated form and a vector which carries at least the pstC2 gene according to SEQ ID No 1, and the use of polynucleotides which comprise the sequences according to the invention as hybridization probes.

### BACKGROUND OF THE INVENTION

The present invention provides nucleotide sequences from coryneform bacteria coding for the pstC2 gene and a process for the fermentative production of amino acids using bacteria in which the pstC2 gene is attenuated. All references cited herein are expressly incorporated by reference. Incorporation by reference is also designated by the term “I.B.R.” following any citation.

L-Amino acids, in particular L-lysine, are used in human medicine and in the pharmaceuticals industry, in the food industry and very particularly in animal nutrition.

It is known that amino acids are produced by fermentation of strains of coryneform bacteria, in particular Corynebacterium glutamicum. Due to their great significance, efforts are constantly being made to improve the production process. Improvements to the process may relate to measures concerning fermentation technology, for example stirring and oxygen supply, or to the composition of the nutrient media, such as for example sugar concentration during fermentation, or to working up to yield the product by, for example, ion exchange chromatography, or to the intrinsic performance characteristics of the microorganism itself.

The performance characteristics of these microorganisms are improved using methods of mutagenesis, selection and mutant selection. In this manner, strains are obtained which are resistant to antimetabolites or are auxotrophic for regulatorily significant metabolites and which produce amino acids.

For some years, methods of recombinant DNA technology have likewise been used to improve strains of Corynebacterium which produce L-amino acids by amplifying individual amino acid biosynthesis genes and investigating the effect on amino acid production.

The invention provides novel measures for the improved fermentative production of amino acids.

### BRIEF SUMMARY OF THE INVENTION

Any subsequent mention of L-amino acids or amino acids should be taken to mean one or more amino acids, including the salts thereof, selected from the group comprising L-asparagine, L-threonine, L-serine, L-glutamate, L-glycine, L-alanine, L-cysteine, L-valine, L-methionine, L-isoleucine, L-leucine, L-tyrosine, L-phenylalanine, L-histidine, L-lysine, L-tryptophan and L-arginine. L-Lysine is particularly preferred.

Any subsequent mention of L-lysine or lysine should be taken to mean not only the bases, but also salts, such as for example lysine monohydrochloride or lysine sulfate.

The invention provides an isolated polynucleotide from coryneform bacteria containing a polynucleotide sequence coding for the pstC2 gene and selected from the group

a) polynucleotide which is at least 70% identical to a polynucleotide which codes for a polypeptide containing the amino acid sequence of SEQ ID no. 2,

b) polynucleotide which codes for a polypeptide which contains an amino acid sequence which is at least 70% identical to the amino acid sequence of SEQ ID no. 2,

c) polynucleotide which is complementary to the polynucleotides of a) or b), and

d) polynucleotide containing at least 15 successive nucleotides of the polynucleotide sequence of a), b) or c),

wherein the polypeptide preferably exhibits the activity of the membrane-bound phosphate transport protein pstC2.

The invention also provides the above-stated polynucleotide, wherein it preferably comprises replicable DNA containing:

(i) the nucleotide sequence shown in SEQ ID no. 1, or

(ii) at least one sequence which matches the sequence (i) within the degeneration range of the genetic code, or

(iii) at least one sequence which hybridizes with the complementary sequences to sequences (i) or (ii) and optionally

(iv) functionally neutral sense mutations in (i).

The invention also provides:

a replicable polynucleotide, in particular DNA, containing the nucleotide sequence as shown in SEQ ID no. 1;

a polynucleotide which codes for a polypeptide which contains the amino acid sequence as shown in SEQ ID no. 2;

a vector containing parts of the polynucleotide according to the invention, specifically at least 15 successive nucleotides of the claimed sequence,

and coryneform bacteria, in which the pstC2 gene is attenuated, in particular by an insertion or deletion.

The invention also provides polynucleotides which substantially consist of a polynucleotide sequence, which are obtainable by screening by means of hybridization of a suitable gene library of a coryneform bacterium, which library contains the complete gene or parts thereof, with a probe which contains the sequence of the polynucleotide according to the invention according to SEQ ID no. 1, or a fragment thereof, and isolation of the stated polynucleotide sequence.

### DETAILED DESCRIPTION OF THE INVENTION

Polynucleotides containing the sequences according to the invention are suitable as hybridization probes for RNA, cDNA and DNA in order to isolate nucleic acids or polynucleotides or full length genes which code for the membrane-bound phosphate transport protein pstC2, or to isolate such nucleic acids or polynucleotides or genes which exhibit a high level of similarity with the sequence of the pstC2 gene. They are also suitable for incorporation into “arrays”, “micro-arrays” or “DNA chips” for the purpose of detecting and determining the corresponding polynucleotides.

Polynucleotides containing the sequences according to the invention are furthermore suitable as primers which may be used, with the assistance of the polymerase chain reaction (PCR), to produce DNA of genes which code for the membrane-bound phosphate transport protein pstC2.

Such oligonucleotides acting as probes or primers contain at least 25, 26, 27, 28, 29 or 30, preferably at least 20, 21, 22, 23 or 24, very particularly preferably at least 15, 16, 17, 18 or 19 successive nucleotides. Oligonucleotides having a length of at least 31, 32, 33, 34, 35, 36, 37, 38, 39 or 40, or at least 41, 42, 43, 44, 45, 46, 47 48, 49 or 50 nucleotides are also suitable. Oligonucleotides having a length of at least 100, 150, 200, 250 or 300 nucleotides are optionally also suitable.

“Isolated” means separated from its natural environment.

“Polynucleotide” generally relates to polyribonucleotides and polydeoxyribonucleotides, wherein the RNA or DNA may be unmodified or modified.

The polynucleotides according to the invention include a polynucleotide according to SEQ ID no. 1 or a fragment produced therefrom and also those which are at least 70% to 80%, preferably at least 81% to 85%, particularly preferably at least 86% to 90% and very particularly preferably at least 91%, 93%, 95%, 97% or 99% identical to the polynucleotide according to SEQ ID no. 1 or a fragment produced therefrom.

“Polypeptides” are taken to mean peptides or proteins which contain two or more amino acids connected by peptide bonds.

The polypeptides according to the invention include a polypeptide according to SEQ ID no. 2, in particular those having the biological activity of the membrane-bound phosphate transport protein pstC2 and also those which are at least 70% to 80%, preferably at least 81% to 85%, particularly preferably at least 86% to 90% and very particularly preferably at least 91%, 93%, 95%, 97% or 99% identical to the polypeptide according to SEQ ID no.2 and exhibit the stated activity.

The invention furthermore relates to a process for the fermentative production of amino acids, selected from the group L-asparagine, L-threonine, L-serine, L-glutamate, L-glycine, L-alanine, L-cysteine, L-valine, L-methionine, L-isoleucine, L-leucine, L-tyrosine, L-phenylalanine, L-histidine, L-lysine, L-tryptophan and L-arginine using coryneform bacteria which in particular already produce amino acids and in which the nucleotide sequences coding for the pstC2 gene are attenuated, in particular suppressed or expressed at a low level.

In this connection, the term “attenuation” means reducing or suppressing the intracellular activity of one or more enzymes (proteins) in a microorganism, which enzymes are coded by the corresponding DNA, for example by using a weak promoter or a gene or allele which codes for a corresponding enzyme which has a low activity or inactivates the corresponding gene or enzyme (protein) and optionally by combining these measures.

The attenuation measures reduce the activity or concentration of the corresponding protein in general to 0 to 75%, 0 to 50%, 0 to 25%, 0 to 10% or 0 to 5% of the activity or concentration of the wild type protein, or the activity or concentration of the protein in the starting microorganism.

The microorganisms, provided by the present invention, may produce amino acids from glucose, sucrose, lactose, fructose, maltose, molasses, starch, cellulose or from glycerol and ethanol. The microorganisms may comprise representatives of the coryneform bacteria in particular of the genus Corynebacterium. Within the genus Corynebacterium, the species Corynebacterium glutamicum may in particular be mentioned, which is known in specialist circles for its ability to produce L-amino acids.

Suitable strains of the genus Corynebacterium, in particular of the species Corynebacterium glutamicum (C. glutamicum), are especially the known wild type strains

Corynebacterium glutamicum ATCC13032

Corynebacterium acetoglutamicum ATCC15806

Corynebacterium acetoacidophilum ATCC13870

Corynebacterium melassecola ATCC17965

Corynebacterium thermoaminogenes FERM BP-1539

Brevibacterium flavum ATCC14067

Brevibacterium lactofermentum ATCC13869 and

Brevibacterium divaricatum ATCC14020

and L-amino acid producing mutants or strains produced therefrom.

The novel pstC2 gene which codes for the membrane-bound phosphate transport protein pstC2 from C. glutamicum was isolated.

The pstC2 gene or also other genes from C. glutamicum are isolated by initially constructing a gene library of this microorganism in Escherichia coli (E. coli). The construction of gene libraries is described in generally known textbooks and manuals. Examples which may be mentioned are the textbook by Winnacker, Gene und Klone, Eine Einführung in die Gentechnologie (Verlag Chemie, Weinheim, Germany, 1990) I.B.R. or the manual by Sambrook et al., Molecular Cloning, A Laboratory Manual (Cold Spring Harbor Laboratory Press, 1989) I.B.R. One very well known gene library is that of E. coli K-12 strain W3110, which was constructed by Kohara et al. (Cell 50, 495-508 (1987)) I.B.R. in λ-vectors. Bathe et al. (Molecular and General Genetics, 252:255-265, 1996) I.B.R. describe a gene library of C. glutamicum ATCC13032, which was constructed using the cosmid vector SuperCos I (Wahl et al., 1987, Proceedings of the National Academy of Sciences USA, 84:2160-2164) I.B.R. in E. coli K-12 strain NM554 (Raleigh et al., 1988, Nucleic Acids Research 16:1563-1575) I.B.R.

Börmann et al. (Molecular Microbiology 6(3), 317-326 (1992)) I.B.R. also describe a gene library of C. glutamicum ATCC 13032, using cosmid pHC79 (Hohn and Collins, 1980, Gene 11, 291-298) I.B.R.

A gene library of C. glutamicum in E. coli may also be produced using plasmids such as pBR322 (Bolivar, 1979, Life Sciences, 25, 807-818) I.B.R. or pUC9 (Vieira et al., 1982, Gene, 19:259-268) I.B.R. Suitable hosts are in particular those E. coli strains with restriction and recombination defects, such as for example strain DH5αmcr, which has been described by Grant et al. (Proceedings of the National Academy of Sciences USA, 87 (1990) 4645-4649) I.B.R. The long DNA fragments cloned with the assistance of cosmids or other λ-vectors may then in turn be sub-cloned in usual vectors suitable for DNA sequencing and then be sequenced, as described, for example, in Sanger et al. (Proceedings of the National Academy of Sciences of the United States of America, 74:5463-5467, 1977) I.B.R.

The resultant DNA sequences may then be investigated using known algorithms or sequence analysis programs, such as for example Staden's program (Nucleic Acids Research 14, 217-232 (1986)) I.B.R., Marck's program (Nucleic Acids Research 16, 1829-1836 (1988)) I.B.R. or Butler's GCG program (Methods of Biochemical Analysis 39, 74-97 (1998)) I.B.R.

The novel DNA sequence of C. glutamicum which codes for the pstC2 gene was found and is provided by the present invention as SEQ ID no. 1. The amino acid sequence of the corresponding protein was furthermore deduced from the above DNA sequence using the methods described above. SEQ ID no. 2 shows the resultant amino acid sequence of the product of the pstC2 gene.

Coding DNA sequences arising from SEQ ID no. 1 due to the degeneracy of the genetic code are also provided by the invention. DNA sequences which hybridize with SEQ ID no. 1 or parts of SEQ ID no. 1 are similarly provided by the invention. Conservative substitutions of amino acids in proteins, for example the substitution of glycine for alanine or of aspartic acid for glutamic acid, are known in specialist circles as “sense mutations”, which result in no fundamental change in activity of the protein, i.e. they are functionally neutral. It is furthermore known that changes to the N and/or C terminus of a protein do not substantially impair or may even stabilize the function thereof. The person skilled in the art will find information in this connection inter alia in Ben-Bassat et al. (Journal of Bacteriology 169:751-757 (1987)) I.B.R., in O'Regan et al. (Gene 77:237-251 (1989)) I.B.R., in Sahin-Toth et al. (Protein Sciences 3:240-247 (1994)) I.B.R., in Hochuli et al. (Bio/Technology 6:1321-1325 (1988)) I.B.R. and in known textbooks of genetics and molecular biology. Amino acid sequences arising in a corresponding manner from SEQ ID no. 2 are also provided by the invention.

DNA sequences which hybridize with SEQ ID no. 1 or parts of SEQ ID no. 1 are similarly provided by the invention. Finally, DNA sequences produced by the polymerase chain reaction (PCR) using primers obtained from SEQ ID no. 1 are also provided by the invention. Such oligonucleotides typically have a length of at least 15 nucleotides.

The person skilled in the art may find instructions for identifying DNA sequences by means of hybridization inter alia in the manual “The DIG System Users Guide for Filter Hybridization” from Boehringer Mannheim GmbH (Mannheim, Germany, 1993) I.B.R. and in Liebl et al. (International Journal of Systematic Bacteriology 41: 255-260 (1991)) I.B.R. Hybridization proceeds under stringent conditions, i.e. the only hybrids to be formed are those in which the probe and target sequence, i.e. the polynucleotides treated with the probe, are at least 70% identical. It is known that the stringency of hybridization, including the washing stages, is influenced or determined by varying buffer composition, temperature and salt concentration. The hybridization reaction is preferably performed at relatively low stringency in comparison with the washing stages (Hybaid Hybridisation Guide, Hybaid Limited, Teddington, UK, 1996) I.B.R.

A 5× SSC buffer at a temperature of approx. 50° C.-68° C. may, for example, be used for the hybridization reaction. At this stage, probes may also hybridize with polynucleotides which are less than 70% identical to the sequence of the probe. Such hybrids are less stable and are removed by washing under stringent conditions. This may, for example, be achieved by reducing the salt concentration 2× SSC and optionally subsequently 0.5× SSC (The DIG System User's Guide for Filter Hybridisation, Boehringer Mannheim, Mannheim, Germany, 1995) I.B.R., with a temperature of approx. 50° C.-68° C. being set. It is optionally possible to reduce the salt concentration down to 0.1× SSC. By a stepwise increase in hybridization temperature in approx. 1-2° C. steps from 50° C. to 68° C., it is possible to isolate polynucleotide fragments which are, for example, at least 70% or at least 80% or at least 90% to 95% identical to the sequence of the probe used. Further instructions with regard to hybridization are commercially available in “kits” (for example DIG Easy Hyb from Roche Diagnostics GmbH, Mannheim, Germany, catalogue no. 1603558).

The person skilled in the art will find instructions for amplifying DNA sequences by means of the polymerase chain reaction (PCR) inter alia in the textbook by Gait, Oligonucleotide Synthesis: A Practical Approach (IRL Press, Oxford, UK, 1984) I.B.R. and in Newton and Graham: PCR (Spektrum Akademischer Verlag, Heidelberg, Germany, 1994) I.B.R.

It has been found that coryneform bacteria produce amino acids in an improved manner once the pstC2 gene has been attenuated.

Attenuation may be achieved by reducing or suppressing either the expression of the pstC2 gene or the catalytic properties of the enzyme protein. Both measures may optionally be combined.

Reduced gene expression may be achieved by appropriate control of the culture or by genetic modification (mutation) of the signal structures for gene expression. Signal structures for gene expression are, for example, repressor genes, activator genes, operators, promoters, attenuators, ribosome binding sites, the start codon and terminators. The person skilled in the art will find information in this connection for example in patent application WO 96/15246 I.B.R., in Boyd & Murphy (Journal of Bacteriology 170: 5949 (1988)) I.B.R., in Voskuil & Chambliss (Nucleic Acids Research 26: 3548 (1998)) I.B.R., in Jensen & Hammer (Biotechnology and Bioengineering 58: 191 (1998)) I.B.R., in Pátek et al. (Microbiology 142: 1297 (1996)) I.B.R., Vasicova et al. (Journal of Bacteriology 181: 6188 (1999)) I.B.R. and in known textbooks of genetics and molecular biology, such as for example the textbook by Knippers (“Molekulare Genetik”, 6th edition, Georg Thieme Verlag, Stuttgart, Germany, 1995) I.B.R. or by Winnacker (“Gene und Klone”, VCH Verlagsgesellschaft, Weinheim, Germany, 1990) I.B.R.

Mutations which give rise to a change or reduction in the catalytic properties of enzyme proteins are known from the prior art; examples which may be mentioned are the papers by Qiu and Goodman (Journal of Biological Chemistry 272: 8611-8617 (1997)) I.B.R., Sugimoto et al. (Bioscience Biotechnology and Biochemistry 61: 1760-1762 (1997)) I.B.R. and Möckel (“Die Threonindehydratase aus Corynebacterium glutamicum: Aufhebung der allosterischen Regulation und Struktur des Enzyms”, Reports from the Jülich research Center, Jül-2906, ISSN09442952, Jülich, Germany, 1994) I.B.R. Summary presentations may be found in known textbooks of genetics and molecular biology such as, for example, the textbook by Hagemann (“Allgemeine Genetik”, Gustav Fischer Verlag, Stuttgart, 1986) I.B.R.

Mutations which may be considered are transitions, transversions, insertions and deletions. Depending upon the effect of amino acid substitution on enzyme activity, the mutations are known as “missense” mutations or “nonsense” mutations. Insertions or deletions of at least one base pair (bp) in a gene give rise to frame shift mutations, as a result of which the incorrect amino acids are inserted or translation terminates prematurely. Deletions of two or more codons typically result in a complete breakdown of enzyme activity. Instructions for producing such mutations belong to the prior art and may be found in known textbooks of genetics and molecular biology, such as for example the textbook by Knippers (“Molekulare Genetik”, 6th edition, Georg Thieme Verlag, Stuttgart, Germany, 1995) I.B.R., by Winnacker (“Gene und Klone”, VCH Verlagsgesellschaft, Weinheim, Germany, 1990) I.B.R. or by Hagemann (“Allgemeine Genetik”, Gustav Fischer Verlag, Stuttgart, 1986) I.B.R.

One common method of mutating genes of C. glutamicum is the method of gene disruption and gene replacement described by Schwarzer & Pühler (Bio/Technology 9, 84-87 (1991)) I.B.R.

In the gene disruption method, a central portion of the coding region of the gene under consideration is cloned into a plasmid vector which may replicate in a host (typically E. coli), but not in C. glutamicum. Vectors which may be considered are, for example, pSUP301 (Simon et al., Bio/Technology 1, 784-791 (1983)) I.B.R., pK18mob or pK19mob (Schäfer et al., Gene 145, 69-73 (1994)) I.B.R., pK18mobsacB or pK19mobsacB (Jäger et al., Journal of Bacteriology 174: 5462-65 (1992) I.B.R.), pGEM-T (Promega Corporation, Madison, Wis., USA), pCR2.1-TOPO (Shuman (1994). Journal of Biological Chemistry 269:32678-84 I.B.R.; U.S. Pat. No. 5,487,993 I.B.R.), pCR®Blunt (Invitrogen, Groningen, Netherlands; Bernard et al., Journal of Molecular Biology, 234: 534-541 (1993) I.B.R.) or pEM1 (Schrumpf et al., 1991, Journal of Bacteriology 173:4510-4516 I.B.R.). The plasmid vector which contains the central portion of the coding region of the gene is then transferred into the desired strain of C. glutamicum by conjugation or transformation. The conjugation method is described, for example, in Schäfer et al. (Applied and Environmental Microbiology 60, 756-759 (1994) I.B.R.). Transformation methods are described, for example, in Thierbach et al. (Applied Microbiology and Biotechnology 29, 356-362 (1988)) I.B.R., Dunican and Shivnan (Bio/Technology 7, 1067-1070 (1989)) I.B.R. and Tauch et al. (FEMS Microbiological Letters 123, 343-347 (1994)) I.B.R. After homologous recombination by means of “crossing over”, the coding region of the gene concerned is interrupted by the vector sequence and two incomplete alleles are obtained, each of which lacks the 3′ or 5′ end. This method has been described, for example by Fitzpatrick et al. (Applied Microbiology and Biotechnology 42, 575-580 (1994)) I.B.R. for suppressing the recA gene of C. glutamicum.

In the gene replacement method, a mutation, such as for example a deletion, insertion of base replacement, is produced in vitro in the gene under consideration. The resultant allele is in turn cloned into a vector which is non-replicative in C. glutamicum, which vector is then transferred into the desired host of C. glutamicum by transformation or conjugation. After homologous recombination by means of a first “crossing over”, which effects integration, and a suitable second “crossing over”, which effects excision, in the target gene or target sequence, the mutation or allele is incorporated. This method has been used, for example by Peters-Wendisch et al. (Microbiology 144, 915-927 (1998)) I.B.R. to suppress the pyc gene of C. glutamicum by a deletion.

A deletion, insertion or base substitution may be incorporated into the pstC2 gene in this manner.

It may additionally be advantageous for the production of L-amino acids, in addition to attenuating the pstC2 gene, to enhance, in particular to overexpress, one or more enzymes of the particular biosynthetic pathway, of glycolysis, of anaplerotic metabolism, of the citric acid cycle, of the pentose phosphate cycle, of amino acid export and optionally regulatory proteins.

For the production of L-amino acids, it is thus possible, in addition to attenuating the pstC2 gene, simultaneously to enhance, in particular overexpress, one or more genes selected from the group

the dapA gene, which codes for dihydropicolinate synthase (EP-B 0 197 335 I.B.R.),

the gap gene, which codes for glyceraldehyde-3-phosphate dehydrogenase (Eikmanns (1992), Journal of Bacteriology 174:6076-6086 I.B.R.),

the tpi gene, which codes for triosephosphate isomerase (Eikmanns (1992), Journal of Bacteriology 174:6076-6086

the pgk gene, which codes for 3-phosphoglycerate kinase (Eikmanns (1992), Journal of Bacteriology 174:6076-6086

the zwf gene, which codes for glucose-6-phosphate dehydrogenase (JP-A-09224661 I.B.R.),

the pyc gene, which codes for pyruvate carboxylase (DE-A-198 31 609 I.B.R.),

the mqo gene, which codes for malate:quinone oxidoreductase (Molenaar et al., European Journal of Biochemistry 254, 395-403 (1998) I.B.R.),

the lysC gene, which codes for feedback resistant aspartate kinase (accession no. P26512),

the lysE gene, which codes for lysine export (DE-A-195 48 222 I.B.R.),

the hom gene, which codes for homoserine dehydrogenase (EPA 0131171 I.B.R.),

the ilvA gene, which codes for threonine dehydratase (Möckel et al., Journal of Bacteriology (1992) 8065-8072) I.B.R.), or the allele ilvA(Fbr) which codes for “feedback resistant” threonine dehydratase (Möckel et al., (1994) Molecular Microbiology 13: 833-84 I.B.R.2),

the ilvBN gene, which codes for acetohydroxy acid synthase (EP-B 0356739 I.B.R.),

the ilvD gene, which codes for dihydroxy acid dehydratase (Sahm and Eggeling (1999) Applied and Environmental Microbiology 65: 1973-1979 I.B.R.),

the gene zwa1, which codes for the Zwa1 protein (DE: 19959328.0 I.B.R., DSM 13115).

For the production of amino acids, it may furthermore be advantageous, in addition to attenuating the pstC2 gene, simultaneously to attenuate, in particular reduce the expression of, one or more genes selected from the group

the pck gene, which codes for phosphoenolpyruvate carboxykinase (DE 199 50 409.1 I.B.R., DSM 13047),

the pgi gene, which codes for glucose-6-phosphate isomerase (U.S. Ser. No. 09/396,478 I.B.R., DSM 12969),

the poxB gene, which codes for pyruvate oxidase (DE: 1995 1975.7 I.B.R., DSM 13114),

the gene zwa2, which codes for the Zwa2 protein (DE: 19959327.2 I.B.R., DSM 13113).

In this connection, the term “enhancement” describes the increase in the intracellular activity of one or more enzymes in a microorganism, which enzymes are coded by the corresponding DNA, for example by increasing the copy number of the gene or genes, by using a strong promoter or a gene which codes for a corresponding enzyme having elevated activity and optionally by combining these measures.

The enhancement, in particular overexpression, measures increase the activity or concentration of the corresponding protein in general by at least 10%, 25%, 50%, 75%, 100%, 150%, 200%, 300%, 400% or 500%, at most by 1000% or 2000%, relative to the activity or concentration of the wild type protein, or the activity or concentration of the protein in the starting microorganism.

It may furthermore be advantageous for the production of amino acids, in addition to attenuating the pstC2 gene, to suppress unwanted secondary reactions (Nakayama: “Breeding of Amino Acid Producing Micro-organisms”, in: Overproduction of Microbial Products, Krumphanzl, Sikyta, Vanek (eds.), Academic Press, London, UK, 1982) I.B.R.

The microorganisms produced according to the invention are also provided by the invention and may be cultured continuously or discontinuously using the batch process or the fed batch process or repeated fed batch process for the purpose of producing L-amino acids. A summary of known culture methods is given in the textbook by Chmiel (Bioprozesstechnik 1. Einführung in die Bioverfahrenstechnik (Gustav Fischer Verlag, Stuttgart, 1991) I.B.R.) or in the textbook by Storhas (Bioreaktoren und periphere Einrichtungen (Vieweg Verlag, Braunschweig/Wiesbaden, 1994)) I.B.R.

The culture medium to be used must adequately satisfy the requirements of the particular strains. Culture media for various microorganisms are described in “Manual of Methods for General Bacteriology” from the American Society for Bacteriology (Washington D.C., USA, 1981) I.B.R.

Carbon sources which may be used include sugars and carbohydrates, such as for example glucose, sucrose, lactose, fructose, maltose, molasses, starch and cellulose, oils and fats, such as for example soya oil, sunflower oil, peanut oil and coconut oil, fatty acids, such as for example palmitic acid, stearic acid and linoleic acid, alcohols, such as for example glycerol and ethanol, and organic acids, such as for example acetic acid. These substances may be used individually or as a mixture.

Nitrogen sources which may be used comprise organic compounds containing nitrogen, such as peptones, yeast extract, meat extract, malt extract, corn steep liquor, soya flour and urea or inorganic compounds, such as ammonium sulfate, ammonium chloride, ammonium phosphate, ammonium carbonate and ammonium nitrate. The nitrogen sources may be used individually or as a mixture.

Phosphorus sources which may be used are phosphoric acid, potassium dihydrogen phosphate or dipotassium hydrogen phosphate or the corresponding salts containing sodium. The culture medium must additionally contain salts of metals, such as magnesium sulfate or iron sulfate for example, which are necessary for growth. Finally, essential growth-promoting substances such as amino acids and vitamins may also be used in addition to the above-stated substances. Suitable precursors may furthermore be added to the culture medium. The stated feed substances may be added to the culture as a single batch or be fed appropriately during culturing.

Basic compounds, such as sodium hydroxide, potassium hydroxide, ammonia or ammonia water, or acidic compounds, such as phosphoric acid or sulfuric acid, are used appropriately to control the pH of the culture. Foaming may be controlled by using antifoaming agents such as fatty acid polyglycol esters for example. Plasmid stability may be maintained by the addition to the medium of suitable selectively acting substances, for example antibiotics. Oxygen or gas mixtures containing oxygen, such as for example air, are introduced into the culture in order to maintain aerobic conditions. The temperature of the culture is normally from 20° C. to 45° C. and preferably from 25° C. to 40° C. The culture is continued until a maximum quantity of the desired product has been formed. This aim is normally achieved within 10 to 160 hours.

Methods for determining L-amino acids are known from the prior art. Analysis may proceed, for example, by anion exchange chromatography with subsequent ninhydrin derivation, as described in Spackman et al. (Analytical Chemistry, 30, (1958), 1190) I.B.R. or by reversed phase HPLC, as described in Lindroth et al. (Analytical Chemistry (1979) 51: 1167-1174) I.B.R.

The purpose of the process according to the invention is the fermentative production of amino acids.

The present invention is illustrated in greater detail by the following practical Examples.

Isolation of plasmid DNA from Escherichia coli and all restriction, Klenow and alkaline phosphatase treatment techniques were performed in accordance with Sambrook et al. (Molecular Cloning. A Laboratory Manual (1989) Cold Spring Harbor Laboratory Press, Cold Spring Harbor, N.Y., USA) I.B.R. Methods for transforming Escherichia coli are also described in this manual.

The composition of usual nutrient media such as LB or TY medium may also be found in the manual by Sambrook et al.

### EXAMPLE 1

Production of a Genomic Cosmid Gene Library from C. glutamicum ATCC 13032

Chromosomal DNA from C. glutamicum ATCC 13032 was isolated as described in Tauch et al., (1995, Plasmid 33:168-179) I.B.R. and partially cleaved with the restriction enzyme Sau3AI (Amersham Pharmacia, Freiburg, Germany, product description Sau3AI, code no. 27-0913-02). The DNA fragments were dephosphorylated with shrimp alkaline phosphatase (Roche Molecular Biochemicals, Mannheim, Germany, product description SAP, code no. 1758250). The DNA of cosmid vector SuperCos1 (Wahl et al. (1987), Proceedings of the National Academy of Sciences USA 84:2160-2164 I.B.R.), purchased from Stratagene (La Jolla, USA, product description SuperCos1 Cosmid Vector Kit, code no. 251301) was cleaved with the restriction enzyme XbaI (Amersham Pharmacia, Freiburg, Germany, product description XbaI, code no. 27-0948-02) and also dephosphorylated with shrimp alkaline phosphatase.

The cosmid DNA was then cleaved with the restriction enzyme BamHI (Amersham Pharmacia, Freiburg, Germany, product description BamHI, code no. 27-0868-04). Cosmid DNA treated in this manner was mixed with the treated ATCC 13032 DNA and the batch was treated with T4 DNA ligase (Amersham Pharmacia, Freiburg, Germany, product description T4 DNA Ligase, code no. 27-0870-04). The ligation mixture was then packed in phages using Gigapack II XL Packing Extracts (Stratagene, La Jolla, USA, product description Gigapack II XL Packing Extract, code no. 200217).

E. coli strain NM554 (Raleigh et al. 1988, Nucleic Acid Res. 16:1563-1575 I.B.R.) was infected by suspending the cells in 10 mM MgSO₄ and mixing them with an aliquot of the phage suspension. The cosmid library was infected and titred as described in Sambrook et al. (1989, Molecular Cloning: A Laboratory Manual, Cold Spring Harbor I.B.R.), the cells being plated out on LB agar (Lennox, 1955, Virology, 1:190 I.B.R.) +100 μg/ml of ampicillin. After overnight incubation at 37° C., individual recombinant clones were selected.

### EXAMPLE 2

Isolation and sequencing of the pstC2 Gene

Cosmid DNA from an individual colony was isolated in accordance with the manufacturer's instructions using the Qiaprep Spin Miniprep Kit (product no. 27106, Qiagen, Hilden, Germany) and partially cleaved with the restriction enzyme Sau3AI (Amersham Pharmacia, Freiburg, Germany, product description Sau3AI, product no. 27-0913-02). The DNA fragments were dephosphorylated with shrimp alkaline phosphatase (Roche Molecular Biochemicals, Mannheim, Germany, product description SAP, product no. 1758250). Once separated by gel electrophoresis, the cosmid fragments of a size of 1500 to 2000 bp were isolated using the QiaExII Gel Extraction Kit (product no. 20021, Qiagen, Hilden, Germany).

The DNA of the sequencing vector pZero-1 purchased from Invitrogen (Groningen, Netherlands, product description Zero Background Cloning Kit, product no. K2500-01) was cleaved with the restriction enzyme BamHI (Amersham Pharmacia, Freiburg, Germany, product description BamHI, product no. 27-0868-04). Ligation of the cosmid fragments into the sequencing vector pZero-1 was performed as described by Sambrook et al. (1989, Molecular Cloning: A Laboratory Manual, Cold Spring Harbor I.B.R.), the DNA mixture being incubated overnight with T4 ligase (Pharmacia Biotech, Freiburg, Germany). This ligation mixture was then electroporated into the E. coli strain DH5αMCR (Grant, 1990, Proceedings of the National Academy of Sciences U.S.A., 87:4645-4649 I.B.R.) (Tauch et al. 1994, FEMS Microbiol Letters, 123:343-7 I.B.R.) and plated out onto LB agar (Lennox, 1955, Virology, 1:190 I.B.R.) with 50 μg/ml of Zeocin.

Plasmids of the recombinant clones were prepared using the Biorobot 9600 (product no. 900200, Qiagen, Hilden, Germany). Sequencing was performed using the dideoxy chain termination method according to Sanger et al. (1977, Proceedings of the National Academies of Sciences U.S.A., 74:5463-5467 I.B.R.) as modified by Zimmermann et al. (1990, Nucleic Acids Research, 18:1067 I.B.R.). The “RR dRhodamin Terminator Cycle Sequencing Kit” from PE Applied Biosystems (product no. 403044, Weiterstadt, Germany) was used. Separation by gel electrophoresis and analysis of the sequencing reaction was performed in a “Rotiphorese NF” acrylamide/bisacrylamide gel (29:1) (product no. A124.1, Roth, Karlsruhe, Germany) using the “ABI Prism 377” sequencer from PE Applied Biosystems (Weiterstadt, Germany).

The resultant raw sequence data were then processed using the Staden software package (1986, Nucleic Acids Research, 14:217-231) I.B.R., version 97-0. The individual sequences of the pzero 1 derivatives were assembled into a cohesive contig. Computer-aided coding range analysis was performed using XNIP software (Staden, 1986, Nucleic Acids Research, 14:217-231 I.B.R.). Further analysis can be performed using the “BLAST search programs” (Altschul et al., 1997, Nucleic Acids Research, 25:33893402 I.B.R.), against the non-redundant database of the “National Center for Biotechnology Information” (NCBI, Bethesda, Md., USA) I.B.R.

The relative degree of substitution or mutation in the polynucleotide or amino acid sequence to produce a desired percentage of sequence identity can be established or determined by well-known methods of sequence analysis. These methods are disclosed and demonstrated in Bishop, et al. “DNA & Protein Sequence Analysis (A Practical Approach”), Oxford Univ. Press, Inc. (1997) I.B.R. and by Steinberg, Michael “Protein Structure Prediction” (A Practical Approach), Oxford Univ. Press, Inc. (1997) I.B.R.

The resultant nucleotide sequence is stated in SEQ ID NO: 1. Analysis of the nucleotide sequence revealed an open reading frame of 1068 base pairs, which was designated the pstC2 gene. The pstC2 gene codes for a polypeptide of 355 amino acids.

This application claims priority to German Priority Document Application No. 100 45 486.0, filed on Sep. 14, 2000. The German Priority Document is hereby incorporated by reference in its entirety.

We claim:

1. An isolated polynucleotide from coryneform bacteria containing a polynucleotide sequence coding for the pstC2 gene and selected from the group consisting of:
a) a polynucleotide which is at least 70% identical to a polynucleotide which codes for a polypeptide containing the amino acid sequence of SEQ ID NO: 2,
b) a polynucleotide which codes for a polypeptide which contains an amino acid sequence which is at least 70% identical to the amino acid sequence of SEQ ID NO: 2,
c) a polynucleotide which is complementary to the polynucleotides of a) or b), and
d) a polynucleotide containing at least 15 successive nucleotides of the polynucleotide sequence of a), b) or c).

2. The polynucleotide according to claim 1, wherein the polypeptide has membrane-bound phosphate transport protein pstC2 activity.

3. The polynucleotide according to claim 1, wherein the polynucleotide is a recombinant DNA replicable in coryneform bacteria.

4. The polynucleotide according to claim 1, wherein the polynucleotide is an RNA.

5. The polynucleotide according to claim 3, comprising the nucleotide sequence as shown in SEQ ID NO: 1.

6. The polynucleotide according to claim 3, wherein the DNA, comprises
(i) the nucleotide sequence shown in SEQ ID NO: 1, or
(ii) at least one sequence which matches the sequence (i) within the degeneration range of the genetic code, or
(iii) at least one sequence which hybridises with the complementary sequence to sequence (i) or (ii).

7. The polynucleotide according to claim 6, further comprising
(iv) functionally neutral sense mutations in (i).

8. The polynucleotide according to claim 6, wherein the hybridization of sequence (iii) is carried out under conditions of stringency corresponding at most to 2×SSC.

9. A polynucleotide sequence according to claim 1, wherein the polynucleotide codes for a polypeptide that comprises the amino acid sequence shown in SEQ ID NO: 2.

10. A coryneform bacteria, in which the pstC2 gene is attenuated or suppressed.

11. A method for the fermentative production of L-amino acids, in coryneform bacteria, comprising:
a) fermenting, in a medium, coryneform bacteria producing the desired L-amino acid, in which at least the pstC2 gene or nucleotide sequences coding therefor is/are attenuated or suppressed.

13. The method according to claim 12, further comprising:
b) concentrating the L-amino acid in the medium or in the cells of the bacteria.

14. The method according to claim 13, further comprising:
c) isolating the L-amino acid.

15. The method according to claim 12, wherein the L amino acids are lysine.

16. The method according to claim 12, wherein additional genes of the biosynthesis pathway of the desired L-amino acid are enhanced in the bacteria.

17. The method according to claim 12, wherein bacteria are used in which the metabolic pathways which reduce the formation of the desired L-amino acid(s) are at least partially suppressed.

18. The method according to claim 12, wherein the expression of the polynucleotide(s) which codes/code for the pstC2 gene is/are attenuated or suppressed.

19. The method according to claim 12, wherein the catalytic properties of the polypeptide, for which the polynucleotide pstC2 codes, are reduced.

20. The method according to claim 12, wherein the bacteria being fermented comprise, at the same time, one or more genes which are enhanced or overexpressed; wherein the one or more genes is/are selected from the group consisting of:
the gene dapA, which codes for dihydropicolinate synthase,
the gap gene, which codes for glyceraldehyde 3-phosphate dehydrogenase,
the tpi gene, which codes for triosephosphate isomerase,
the pgk gene, which codes for 3-phosphoglycerate kinase,
the zwf gene, which codes for glucose-6-phosphate dehydrogenase,
the pyc gene, which codes for pyruvate carboxylase,
the mqo gene, which codes for malate:quinone oxidoreductase,
the lysc gene, which codes for feedback resistant aspartate kinase,
the lysE gene, which codes for lysine export,
the hom gene, which codes for homoserine dehydrogenase,
the ilvA gene, which codes for threonine dehydratase, or the allele ilvA(Fbr), which codes for feedback resistant threonine dehydratase,
the ilvBN gene, which codes for acetohydroxy acid synthase,
the ilvD gene, which codes for dihydroxy acid dehydratase, and
the gene zwa1, which codes for the Zwa1 protein.

21. The method according to claim 12, wherein the bacteria being fermented comprise, at the same time, one or more genes which are attenuated; wherein the genes are selected from the group consisting of:
the pck gene, which codes for phosphoenolpyruvate carboxykinase,
the pgi gene, which codes for glucose-6-phosphate isomerase,
the poxB gene, which codes for pyruvate oxidase, and
the gene zwa2, which codes for the Zwa2 protein.

22. A method according to claim 12, wherein microorganisms of the species Corynebacterium glutamicum are used.

23. A Coryneform bacteria comprising a vector which includes portions of the polynucleotide according to claim 1.

24. The Coryneform bacteria according to claim 23 wherein the vector includes at least 15 successive nucleotides of the polynucleotide.

25. A method for identifying RNA, cDNA and DNA in order to isolate nucleic acids or polynucleotides or genes which code for the membrane-bound phosphate transport protein pstC2 or exhibit a high level of similarity to the sequence of the pstC2 gene, comprising contacting the RNA, cDNA, or DNA with hybridization probes comprising polynucleotide sequences according to claim 1.

26. The method according to claim 25, wherein arrays, micro-arrays or DNA chips are used.
